(19) United States
(12) Patent Application Publication
(10) Pub. No.: US 2013/0204247 A1
(43) Pub. Date: Aug. 8, 2013

(54) Ergonomic Handle for Surgical Instrument

(71) Applicant: IMDS Corporation, Logan, UT (US)

(72) Inventor: Daniel J. Triplett, Providence, UT (US)

(73) Assignee: IMDS CORPORATION, Logan, UT (US)

(21) Appl. No.: 13/761,019

(22) Filed: Feb. 6, 2013

Related U.S. Application Data
(60) Provisional application No. 61/595,209, filed on Feb. 6, 2012.

(51) Int. Cl. A61B 17/00

(52) U.S. Cl. CPC A61B 17/00234
USPC 606/53

(57) ABSTRACT

A surgical instrument handle includes adaptations for improved ergonomics, such as broad, rounded hand-contacting surfaces and physiologic range of motion for instrument actuation. The handle may be mostly enclosed by the hand in use, and may be stabilized by the palm, ring finger, and little finger. One example includes a ratchet mechanism and release button.

### CROSS-REFERENCE TO RELATED APPLICATIONS

This application claims the benefit of:

U.S. Application No. 61/595,209, filed Feb. 6, 2012, entitled ERGONOMIC HANDLE FOR SURGICAL INSTRUMENT, Attorney's docket no. MLI-107 PROV, which is pending.

The above-referenced document is hereby incorporated by reference in its entirety.

### BACKGROUND

The present disclosure relates to instrument handle designs for improved ergonomics. Specifically, the described embodiments may be incorporated into surgical instruments, such as rongeurs, graspers, cutters, and the like. It will be appreciated that the disclosed embodiment may have application to other instruments or tools for surgical, medical, or non-medical uses.

### BRIEF DESCRIPTION OF THE DRAWINGS

While exemplary embodiments of the present technology have been shown and described in detail below, it will be clear to the person skilled in the art that variations, changes and modifications may be made without departing from its scope. As such, that which is set forth in the following description and accompanying drawings is offered by way of illustration only and not as a limitation. The actual scope of the invention is intended to be defined by the following claims, along with the full range of equivalents to which such claims are entitled.

In the following Detailed Description, various features are grouped together in several embodiments for the purpose of streamlining the disclosure. This method of disclosure is not to be interpreted as reflecting an intention that exemplary embodiments of the technology require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

Identical reference numerals do not necessarily indicate an identical structure. Rather, the same reference numeral may be used to indicate a similar feature or a feature with similar functionality. Not every feature of each embodiment is labeled in every figure in which that embodiment appears, in order to keep the figures clear. Similar reference numbers (e.g., those that are identical except for the first numeral) are used to indicate similar features in different embodiments.

FIG. 1 is a side view of a prior art surgical instrument;

FIG. 2A is a side view of a surgical instrument in a first position; and FIG. 2B is a side view of the surgical instrument of FIG. 2A in a second position;

FIG. 3A is a side view of another surgical instrument in a first position; and FIG. 3B is a side view of the surgical instrument of FIG. 3A in a second position;

FIG. 4 is a side cross section view of a handle portion of the surgical instrument of FIG. 3A in the first position;

FIG. 5 is a side cross section view of the handle portion of the surgical instrument of FIG. 3A in the second position;

FIG. 6A is a side isometric view of an actuation linkage of the handle portion of the surgical instrument of FIG. 3A in the first position; and FIG. 6B is a side isometric view of the actuation linkage of FIG. 6B in the second position;

FIG. 7 is a side isometric view of a handle body of the handle portion of the surgical instrument of FIG. 3A; and

FIG. 8 is a side isometric view of a lever of the handle portion of the surgical instrument of FIG. 3A;

FIG. 9 is a side view of the prior art surgical instrument of FIG. 1 held by a user in a fingertip grip; and

FIG. 10 is a side view of the prior art surgical instrument of FIG. 1 held by a user in a palm grip.

### DETAILED DESCRIPTION

Standard medical planes of reference and descriptive terminology are employed in this specification. A sagittal plane divides a body into right and left portions. A mid-sagittal plane divides the body into bilaterally symmetric right and left halves. A coronal plane divides a body into anterior and posterior portions. A transverse plane divides a body into superior and inferior portions. Anterior means toward the front of the body. Posterior means toward the back of the body. Superior means toward the head. Inferior means toward the feet. Medial means toward the midline of the body. Lateral means away from the midline of the body. Axial means toward a central axis of the body. Abaxial means away from a central axis of the body.

The following disclosure is made in the context of a manual arthroscopic instrument for the purposes of illustration. The principles of the disclosed technology are applicable to a variety of other tools and instruments outside the scope of manual arthroscopic instruments.

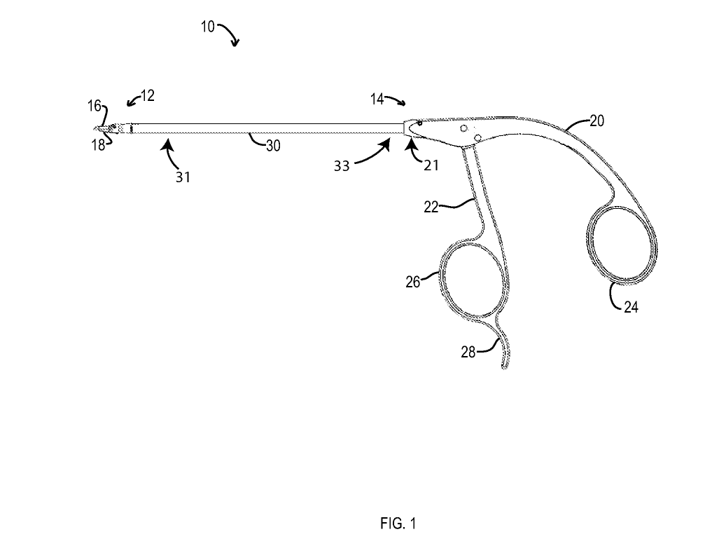

Referring to FIG. 1, a surgical instrument 10 includes a working portion 12 and a handle portion 14. The working portion 12 in this example includes a pair of opposed jaws 16, 18 for grasping or biting a substance such as tissue. Other working portions and/or end effectors may be substituted: rongeurs, scissors, suture graspers, suture passers, knot tiers, implant manipulators, implant actuators, and the like. The handle portion 14 includes a handle body 20 and a lever 22 mounted to the handle body 20. The handle body 20 includes a finger loop 24. The lever 22 includes a finger loop 26 and a finger rest 28.

The working portion 12 is operatively assembled to the handle portion 14 so that movement of the lever 22 relative to the handle body 20 causes actuation of the working portion 12. In this example, the working portion 12 is carried by, or supported by, the handle body 20. The lever 22 is pivotally mounted to the handle body 20, and is connected to at least one sub-component of the working portion 12, as will be discussed in more detail below. As the lever 22 pivots relative to the handle body 20, the working portion 12 is actuated. In this example, the jaws 16, 18 open and close as the lever 22 pivots relative to the handle body 20. The jaws 16, 18 may be said to move between a first state and a second state as the lever 22 pivots relative to the handle body 20. In other examples, a different working portion may perform a different action in response to movement of the lever 22. Some examples of basic actions are opening, sliding, rotating, protruding, locking, cutting, vibrating, oscillating, reciprocating, heating, electrifying, magnetizing, illuminating, imaging, and the like. Each action and its opposite action may be considered a pair of first and second states. It will be appreciated that, in some examples, multiple actions may occur in response to movement of the lever 22 in one direction, and their opposites may occur in response to movement of the lever 22 in an opposite second direction.

In one specific example, a stationary jaw, such as jaw 18, may be coupled to a first end 31 of a tube 30. An opposite second end 33 of the tube 30 may be coupled to a first end 21 of the handle body 20. A movable jaw, such as jaw 16, may be opposably hinged to the stationary jaw 18, and may also be connected to a first end of a shaft 32 (not shown), the first end of the shaft 32 adjacent the first end 31 of the tube 30. The shaft 32 may be slidably and/or rotatably received within the tube 30. An opposite second end of the shaft 32 may be connected to the lever 22, the second end of the shaft 32 adjacent the first end 21 of the handle body 20. The lever 22 may be pivotally mounted to the handle body 20. In this example, when the lever 22 is pivoted toward the handle body 20 as shown in FIG. 1, the jaws 16, 18 close. When the lever 22 is pivoted away from the handle body 20 (not shown), the jaws 16, 18 open.

The arrangement of the handle portion 14 may permit one or more of a user's digits (fingers) to be inserted into the finger loops of the handle body 20 and lever 22, as shown in FIG. 9. For example, an index finger 990 may be inserted into the lever finger loop 26, a middle finger 992 may contact the finger rest 28, and a thumb 998 may be inserted into the handle body finger loop 24. While some users may find this “fingertip grip” to be satisfactory in terms of comfort, instrument control, mechanical advantage, efficiency, and the like, other users may find the fingertip grip to be unsatisfactory for continual use, or unsatisfactory right from the start.

It has been observed that some users avoid inserting their digits into the finger loops. Instead, these users may support the handle body 20 in the palm of the hand, with one or more fingers resting on the lever 22. In other words, the hand wraps around the outside of the handle portion 14. This “palm grip” may be preferred, at least by some users, because the wrist remains in an ergonomically neutral position, the instrument 10 tends to sit lower in the hand, the instrument 10 feels more stable, the fingers are unconfined by the finger loops 24, 26, and/or the instrument 10 is supported by portions of the hand other than the index and/or middle fingers 990, 992, so that support is independent of instrument actuation by these fingers.

The following disclosure sets forth examples of instrument handle portions that may be adapted for the palm grip. The example handle portions are also adapted for actuation by the index and/or middle fingers, which are the so-called power fingers of the hand. The examples may be perceived by users as being better balanced than fingertip grip designs. The examples illustrate that the handle portion may be oriented relative to the working portion so that the user's wrist remains in an ergonomically neutral position as the instruments are being used. An example with a ratchet mechanism is disclosed. The examples may be cost competitive relative to existing fingertip grip instruments.

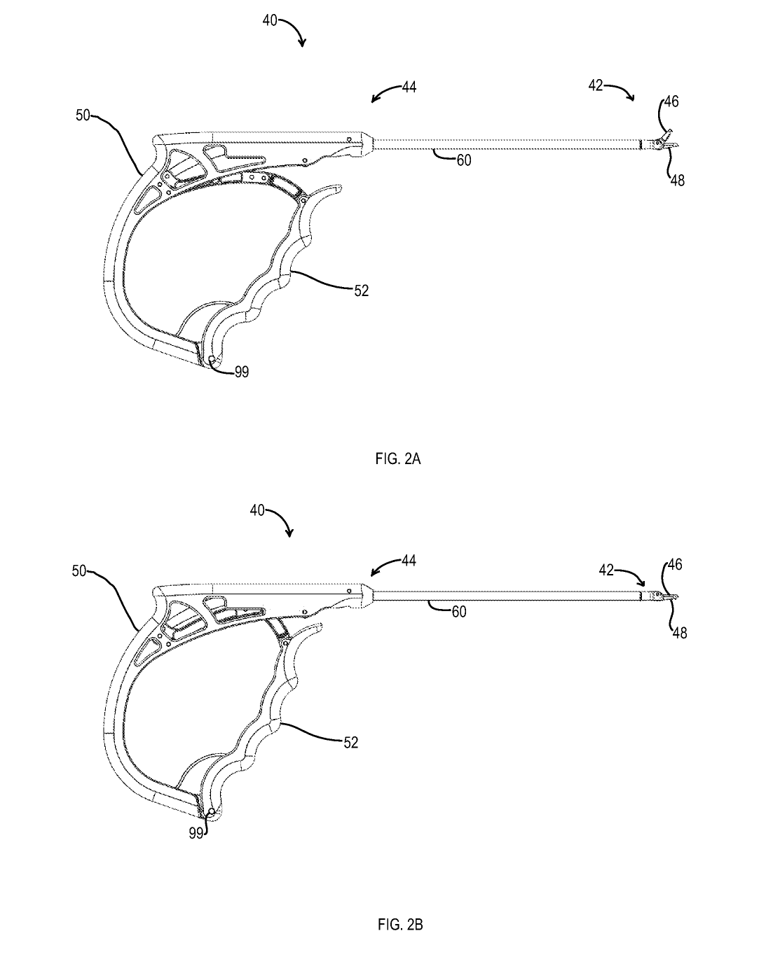

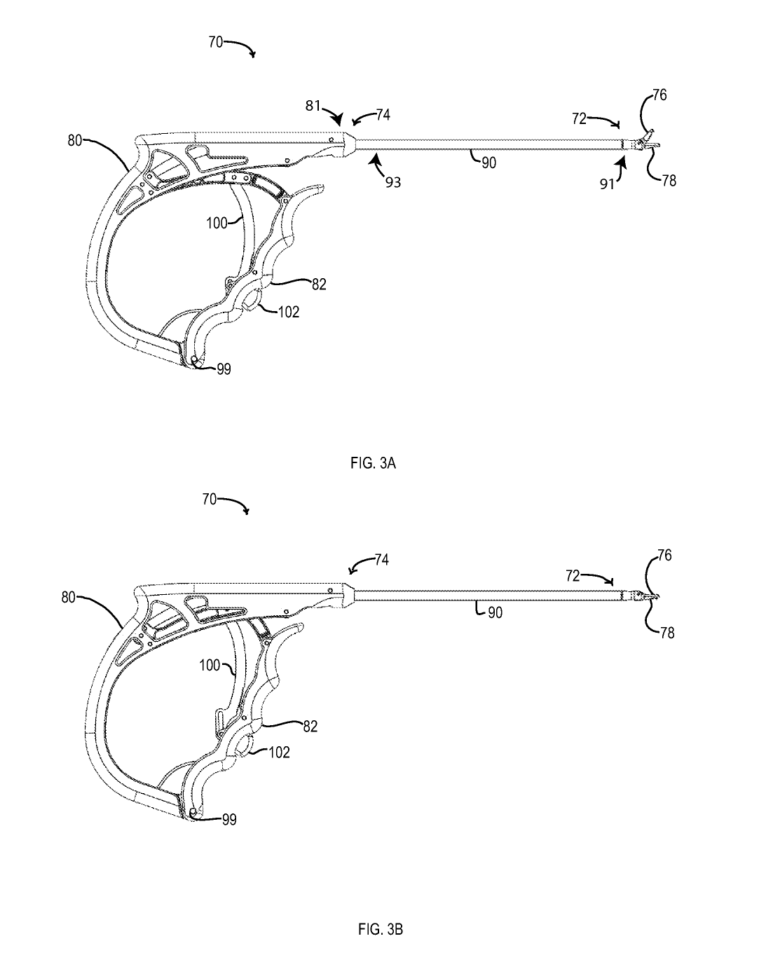

Referring to FIGS. 2A-2B, a surgical instrument 40 includes a working portion 42 and a handle portion 44. The working portion 42 in this example includes a pair of opposed jaws 46, 48 for grasping or biting a substance such as tissue. Other working portions and/or end effectors may be substituted: rongeurs, scissors, suture graspers, suture passers, knot tiers, implant manipulators, implant actuators, and the like. The handle portion 44 includes a handle body 50 and a lever 52 mounted to the handle body 50. The instrument 40 includes a tube 60 between the working portion 42 and the handle portion 44, and a shaft 62 (not shown) inside the tube. The surgical instrument 40 of FIGS. 2A-2B may share some of the characteristics set forth below for the surgical instrument 70 of FIGS. 3A-3B.

The following description for instrument 70 also applies to instrument 40 of FIGS. 2A-2B, except as noted with regard to the ratchet link 100, button 102, button hole 85, and pin hole 88.

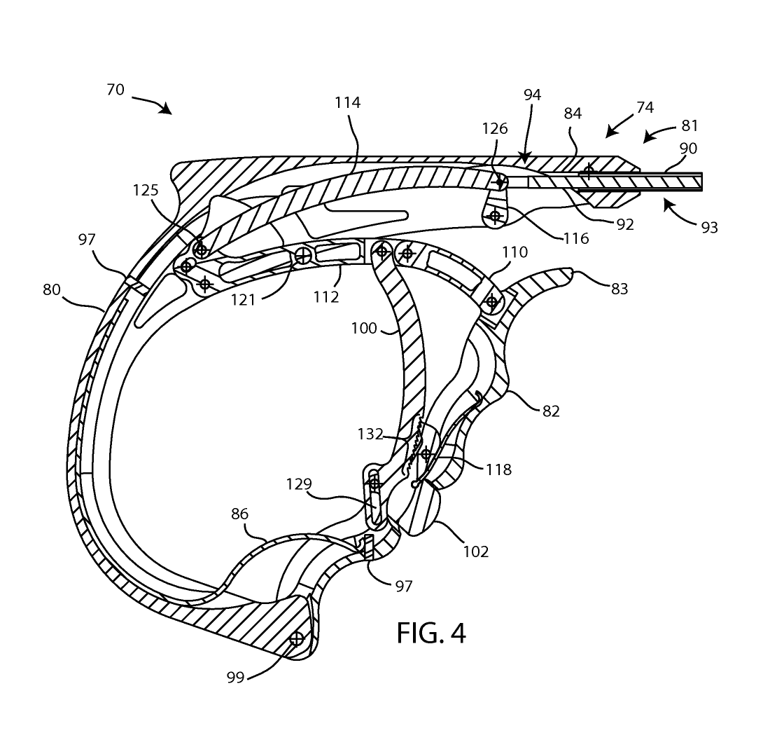

Referring to FIGS. 3A-3B, another surgical instrument 70 includes a working portion 72 and a handle portion 74. The working portion 72 in this example includes a pair of opposed jaws 76, 78 for grasping or biting a substance such as tissue. Other working portions and/or end effectors may be substituted: rongeurs, scissors, suture graspers, suture passers, knot tiers, implant manipulators, implant actuators, and the like. The handle portion 74 includes a handle body 80 and a lever 82 mounted to the handle body 80. The instrument 70 includes a tube 90 between the working portion 72 and the handle portion 74, and a shaft 92 (FIG. 4) inside the tube. The surgical instrument 70 of FIGS. 3A-3B may share some or all of the characteristics set forth above for the surgical instrument 40 of FIGS. 2A-2B. Unlike the surgical instrument of FIGS. 2A-2B, the example of FIGS. 3A-3B includes a ratchet link 100 and a button 102.

In the examples of FIGS. 2A-3B, the working portion is operatively assembled to the handle portion so that movement of the lever relative to the handle body causes actuation of the working portion. In these examples, the working portion is carried by, or supported by, the handle body. The lever may be pivotally mounted to the handle body, and may be connected to at least one sub-component of the working portion. As the lever pivots relative to the handle body, the working portion is actuated. In these examples, the jaws open and close as the lever pivots relative to the handle body. In other examples, the working portion may perform some other action, as listed above, in response to movement of the lever.

In the example of FIGS. 3A-3B, a stationary jaw, such as jaw 78, may be coupled to a first end 91 of a tube 90. An opposite second end 93 of the tube 90 may be coupled to a first end 81 of the handle body 80. In this example, the second end 93 is fixed in a hole 84 of the handle body; thus tube 90 may be described as a stationary shaft. A movable jaw, such as jaw 76, may be opposably hinged to the stationary jaw 78, and may also be connected to a first end (not shown) of a shaft 92 (FIG. 4), where the first end of the shaft 92 is adjacent the first end 91 of the tube 90. The shaft 92 may be slidably and/or rotatably received within the tube 90; thus shaft 92 may be described as a movable shaft. An opposite second end 94 (FIG. 4) of the shaft 92 may be connected to the lever 82, where the second end 94 of the shaft 92 is adjacent the first end 81 of the handle body 80. In this example, the second end 94 of the shaft 92 is coupled to the lever 82 by a series of links, as will be discussed in more detail below.

In another example of the present technology, an outer tube may be the movable shaft and an inner shaft may be the stationary shaft. In yet another example, the stationary and movable shafts may both be tubes. In yet another example, the stationary and movable shafts may lie side by side, and may be solid or tubular. A solid shaft may have one or more inclusions, such as an inner conductive core surrounded by insulation. In further examples, multiple stationary and/or movable shafts may be included.

The handle portion 74 may be shaped and sized to substantially fill the user's palm. The size of the handle portion 74 may take into account published anthropometric data and human factors recommendations for grip span. As one example, the handle portion 74 may have a grip span of 5.5 cm at the lever pivot point (pin 99, FIG. 3A), 7 cm at the center of the lever 82, and 6.5 cm at the lever tip 83. Other grip span dimensions are contemplated in order to fit the natural distribution of hand sizes in the human population. The handle portion 74 may be shaped and sized so that the palm and the lesser two fingers, and optionally the thumb, are used to hold the handle portion 74, leaving the index and middle fingers free to actuate the lever 82.

The lever 82 may be pivotally mounted to the handle body 80, such as by pin 99 as shown in FIG. 3A. Pin 99 may be referred to as a lever pivot point 99. Pin 99 is transversely offset from a central longitudinal axis of the tube 90 by about a handbreadth. The transverse offset may be between 6 cm and 10 cm; other transverse offset dimensions are contemplated in order to fit the natural distribution of hand sizes in the human population, and in order to accommodate different design intents with regard to the amount of handle portion 74 covered by a user's hand. When the lever 82 is pivoted away from the handle body 80, the jaws 76, 78 may be open, as shown in FIG. 3A. When the lever 82 is pivoted close to the handle body 80, the jaws 76, 78 may be closed, as shown in FIG. 3B.

The instrument 70 of FIGS. 3A-3B may include a multi-link mechanism which cooperates with the handle body 80, lever 82, and pin 99 to provide the desired actuation stroke length and force magnification, also known as mechanical advantage. By positioning the lever pivot point 99 at a distance from the central longitudinal axis of the tube 90, each finger moves through an anatomically and ergonomically appropriate actuation stroke length. The longer, stronger index and middle fingers can move farther than the smaller, weaker ring and little fingers.

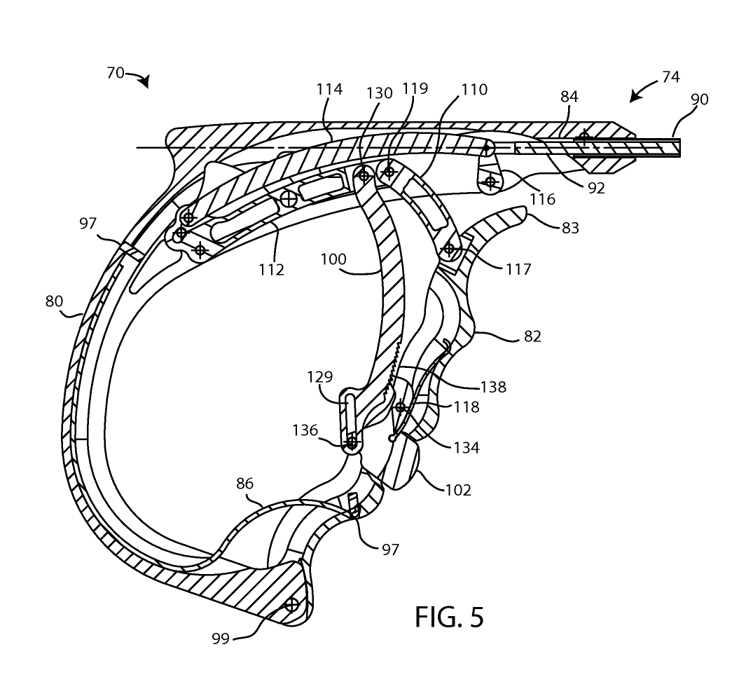

Referring to FIGS. 4-5, the handle portion 74 of the surgical instrument 70 of FIGS. 3A-3B has been cross sectioned along a plane of bilateral symmetry, also known as a center plane, mid-plane, or mid-sagittal plane. FIG. 4 shows the instrument 70 in the open, resting, or non-actuated position and FIG. 5 shows the instrument in the closed, active, or actuated position. While the following description points out specific characteristics of individual component parts, one of skill in the art will recognize that at least some of these characteristics may be altered, varied, or omitted without sacrificing the salient principles of the technology.

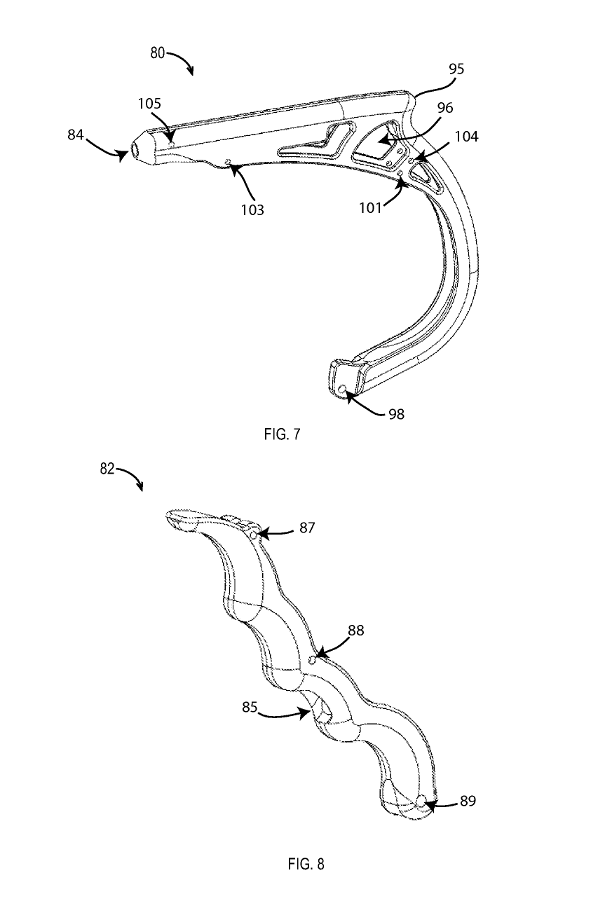

Referring to FIGS. 4-5 and 7, the handle body 80 may be a thin-walled, hollow component that may be the primary structural element of the instrument. An aperture 96 may extend through the handle body 80; this example includes three apertures 96 of various shapes and sizes. The handle body 80 may present broad, rounded surfaces for contact with the palm of the hand. The palm-contacting surfaces, or backstrap, may be textured or may include high friction inserts to improve grip security. An extension, tang, or beavertail 95 may be present. The handle body 80 may be economically manufactured using a casting process, as one example. Secondary operations may be performed, for example after casting, to form a shaft hole 84 and pin holes 98, 101, 103, 104, 105. The handle body 80 may be simplified as a triangle, with apices at the center points of holes 98, 101, 103, and line segments connecting the points. The center point of hole 98 may be referred to as a main pivot point because it forms a main pivot joint in combination with point 89 of the lever 82.

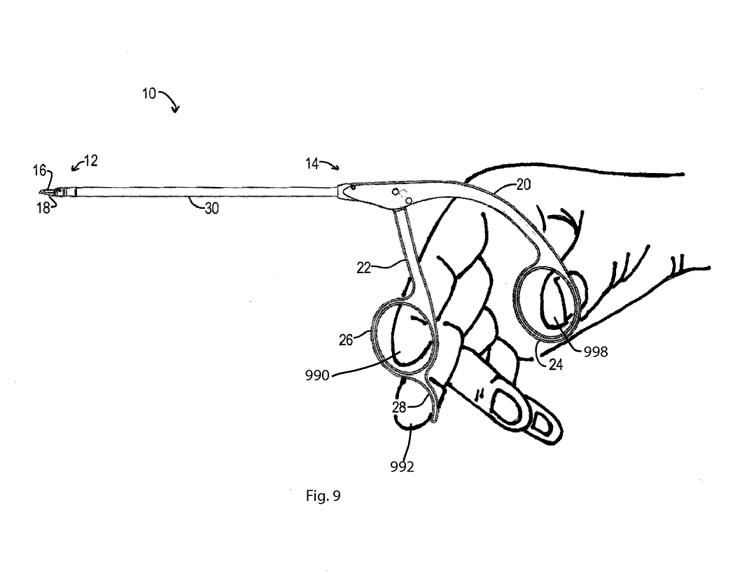

With reference to FIGS. 4-5 and 8, the lever 82 may be a thin-walled, hollow component that is the location where the user applies actuation forces to the instrument 70. The lever 82 may house a ratchet mechanism, as may be seen in FIGS. 3A-5. The lever 82 may present broad, rounded surfaces for contact with the fingers of the hand. The lever 82 may be manufactured using a casting process, as one example. Secondary operations may be performed, for example after casting, to form a button hole 85 and pin holes 87, 88, 89. The lever 82 may be hinged to the handle body 80 by a pin through holes 89 and 98. The lever 82 may be simplified as a line segment extending between the center points of holes 87 and 89.

The lever 52 of the surgical instrument 40 of FIGS. 2A-2B may be similar to lever 82, but may lack the button hole 85 and/or pin hole 88.

With reference to FIGS. 4-5, a main spring 86 may bias the instrument in the open position, although in other examples, the main spring 86 may bias the instrument in a closed position. The main spring 86 may be free-floating within the hollow sections of the handle body 80 and lever 82. The position of the main spring 86 may be constrained in the handle body 80 and lever 82 by a retention pin 97 in each component.

Referring to FIGS. 4-6B, the first link 110 may be a connecting link. The first link 110 connects the lever 82 to the second link 112, described below. The first link 110 may include an aperture 111, and pin holes 117, 119 in opposite ends of the first link 110 for connection to the lever 82 and second link 112, respectively. The first link 110 may be hinged to the lever 82 at a first joint by a pin through holes 87 and 117. The first link 110 may be simplified as a line segment extending between the center points of holes 117, 119.

The second link 112 may be the force magnification link. The second link 112 receives input forces from the first link 110 and transfers the forces to the third link 114. The magnitude of the force magnification, or mechanical advantage, can vary with the motion of the mechanism and/or the dimensions of the second link 112. The magnification can increase as the mechanism moves from the open to the closed position. The second link 112 includes holes 120, 123, 124 for connection to the first link 112, the handle body 80, and the third link 114, respectively. The second link 112 may be hinged to the first link 110 at a second joint by a pin through holes 119 and 120, and hinged to the handle body 80 at a third joint by a pin through holes 101 and 123. The second link 112 may also provide a connection point, or hole 122 for the ratchet link 100 as well as features to constrain the overall range of motion, such as a pin in slot 115 and hole 104, which may form a sliding joint. The second link 112 may include an aperture 113; this example includes two apertures 113. The second link 112 may also include a hole 121 for a drag plug (not shown).

The second link 112 may be simplified as a triangle, with apices at the center points of holes 120, 123, and 124, and line segments connecting the points. The mechanical advantage of the second link 112 may be at least partially determined by dividing the length of the line segment between points 123 and 120 by the length of the line segment between points 123 and 124.

The third link 114 may be another connecting link. The third link 114 connects the second link 112 to the jaw drawbar, or shaft 92. The third link 114 may include pin holes 125, 126 in opposite ends of the third link 114 for connection to the second link 112 and shaft 92, respectively. The third link 114 may be hinged to the second link 112 at a fourth joint by a pin through holes 124 and 125. The third link 114 may be simplified as a line segment extending between the center points of holes 125, 126.

The fourth link 116 may be a control link that modulates, adjusts, or aligns the force vector from the third link 114 to match or align with the center longitudinal axis of the drawbar 92, or a center longitudinal axis of the hole 84 through which the drawbar 92 passes. This may minimize load-induced deflections in the mechanism. The fourth link 116 may include pin holes 127, 128 in opposite ends of the fourth link 116 for connection to the third link 114 and handle body 80, respectively. The fourth link 116 may be hinged to the third link 114 at a fifth joint by a pin through holes 126 and 127, and hinged to the handle body 80 at a sixth joint by a pin through holes 103 and 128. The fourth link 116 may be simplified as a line segment extending between the center points of holes 127, 128.

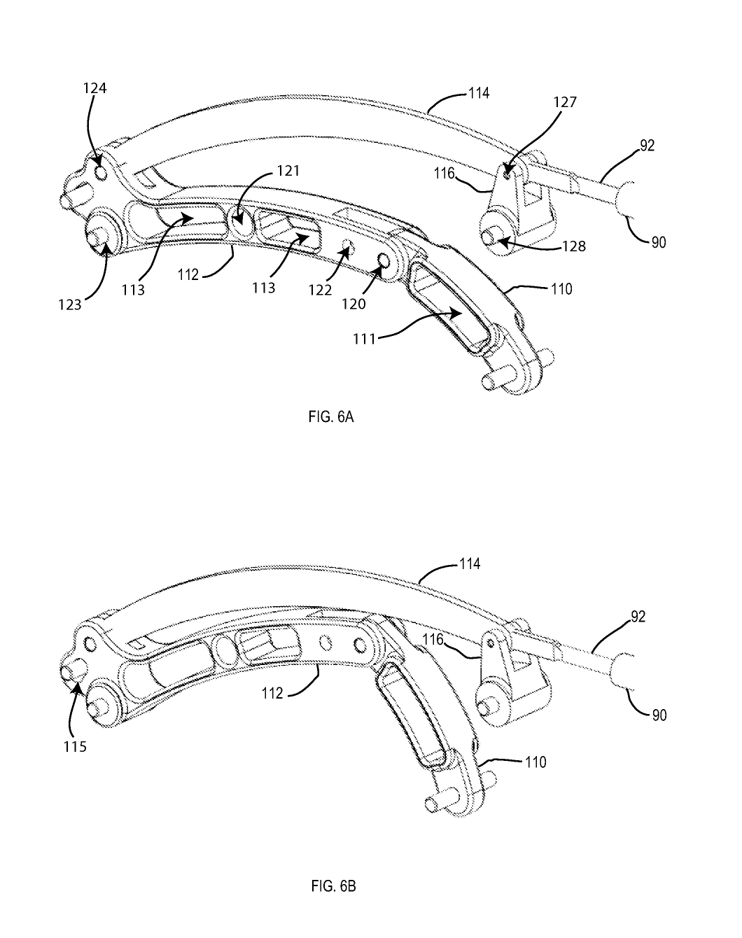

The actuation linkage is shown in the open position in FIGS. 4 and 6A, and in the closed position in FIGS. 5 and 6B. As the lever 82 rotates toward the handle body 80, the center point of hole 126 of the third link 114 moves away from the first end 81 of the handle body 80. The center point of hole 126 of the third link 114 moves along an arcuate path controlled by the fourth link 116. The arcuate path includes a linear component acting along the center longitudinal axis of the hole 84 in the handle body 80 which receives the second end 93 of the tube 90 and/or the second end 94 of the shaft 92, or other instrument components suitable for rongeurs, scissors, suture graspers, suture passers, knot tiers, implant manipulators, implant actuators, and the like. The arcuate path also includes a transverse component acting normal to the center longitudinal axis of the hole 84 in the handle body 80; this transverse component may be a small percentage of the linear component, and in some examples may be zero.

The lever 82 receives an input force/rotation from the hand of a user, and by means of the linkage, transforms the user input to an output force/displacement at the center point of hole 126 of the third link 114. In the example illustrated, the output is primarily a linear force/translation along the center longitudinal axis of the hole 84 in the handle body 80, and secondarily a force/motion acting transverse to the center longitudinal axis of the hole 84. The center point/axis of hole 126 of the third link 114 may be referred to as an actuation point, a drive point, or an output point of the handle portion 74. The center longitudinal axis of the hole 84 may be referred to as an actuation axis, a drive axis, or an output axis of the handle portion 74.

The components of the actuation linkage may be manufactured using traditional machining processes or formed by casting with minimal secondary operations to the pin holes and clevis widths.

Alternate examples of the multi-link mechanism will now be discussed. These alternatives are contemplated for instruments 40 and 70.

The first link 110 may be replaced with a sliding connection between the lever 82 and the second link 112. In this arrangement, lever 82 and second link 112 may be directly connected. The sliding connection may be a pin-in-slot arrangement, as illustrated elsewhere in the mechanism above, or it may be a shaft-in-collar arrangement, or the like. In one example, lever 82 may be modified to include a fixed extension with a forked end for engagement with a pin in hole 120 of the second link 112. The orientation and extent of the fork may be selected to guide the pin in hole 120 along a path between the open and closed positions. The path may be similar or identical to a path between the open and closed positions followed by a pin through holes 119 and 120 in the mechanism of FIGS. 3A-5.

The third link 114 may be replaced with two or more individual links. This arrangement may be useful in situations where the linkage detours around an obstacle, such as another component in the handle portion 74. Some or all of the individual links which replace the third link 114 may be hinged together, or may include sliding connections, or they may bear against an internal part of the handle portion 74, or they may be constrained by one or more control links similar to the fourth link 116.

The fourth link 116 may be replaced with a sliding connection between the third link 114 and the handle body 80. The sliding connection may be like the ones described previously.

The handle portion 74 may also be modified to include a transducer to convert the planar (arcuate, quasi-linear, linear, etc.) motion of the center point of hole 126 of the third link 114 (the output point of the handle portion 74) to rotational movement of a shaft, such as shaft 92. In one example, motion of the output point may be mechanically converted to rotational motion by modifying the handle portion 74 to include an intermediate shaft between the third link 114 and the shaft 92. In this arrangement, the shaft 92 may be proportionately shortened. A first end of the intermediate shaft may be hinged to the third link 114 at hole 126, and optionally hinged to the fourth link 116, if present, at hole 128. An opposite second end of the intermediate shaft may include one or more transverse protrusions, such as posts, tabs, ears, bosses, and the like. The second end 94 of the shaft 92 may be modified to include a socket shaped and sized to receive the second end of the intermediate shaft. The interior of the socket may include one or more helical grooves shaped, sized, and positioned to receive the one or more transverse protrusions; the number of grooves is equal to or greater than the number of transverse protrusions. As the center point of hole 126 of the third link 114 moves along its path in response to movement of the lever 82, the intermediate shaft moves accordingly. The transverse protrusions interact with the helical grooves to force the socket, and therefore shaft 92, to turn. This example provides alternating rotational motion as the handle portion moves between the open and closed positions.

In a further development of this example, a clutch and/or flywheel may be added so that the shaft 92 may be driven in a single rotational direction, and may build up speed with each actuation of the lever 82.

Returning to FIGS. 3A-5, the ratchet link 100 may connect to the second link 112 in the same general area as the first link 110. Hole 130 of the ratchet link 100 is shown connected to hole 122 of the second link 112 by a hinge pin, forming a joint. This connection location exhibits the largest displacement during actuation in this example. The ratchet link 100 may be tied to, or connected to, the ratchet button 102 via a sliding connection of a pin in slot 129 and hole 136 of the ratchet button 102. The ratchet link 100 may include a friction zone 132 with teeth, serrations, grooves, ridges, coating, or other high friction means for discrete or infinitely variable engagement with the button 102, as will be discussed below.

Hole 134 of the ratchet button 102 may be rotationally pinned to hole 88 of the lever 82 at a joint. Hole 136 of the ratchet button 102 may carry a pin for connection to the ratchet link 100 via sliding joint with slot 129. The button 102 may include a friction zone 138 with teeth, serrations, grooves, ridges, coating, or other high friction means for discrete or infinitely variable engagement with the ratchet link 100. The button 102 may be spring biased to protrude out from the front of the lever 82 through hole 85. As the button 102 is depressed, the pin in the sliding joint pushes the ratchet link 100 away from the button 102, separating the friction zones 132, 138, which are mating tooth patterns in this example.

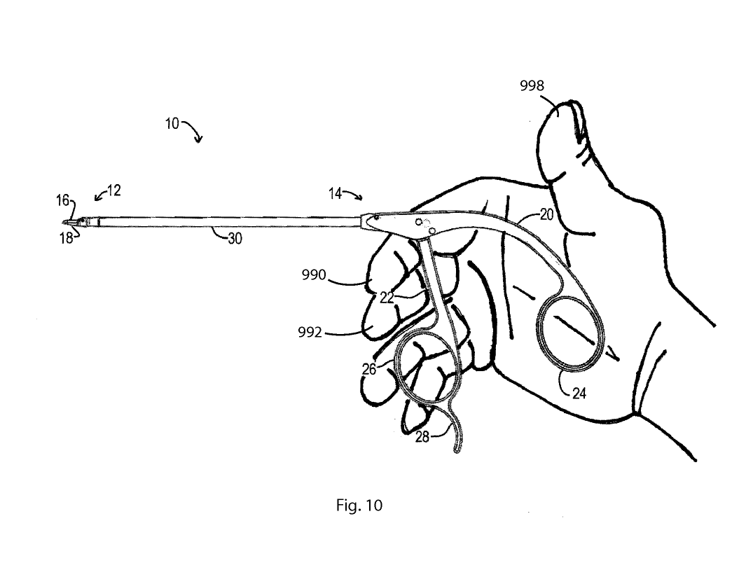

When the handle portion 74 is grasped by a hand of a user, the output axis is adjacent a thumb of the hand, the main pivot joint is adjacent a small finger of the hand, and a finger of the hand may rest on the button. This is similar to the palm grip illustrated in FIG. 10. With reference to FIG. 3A, it will be appreciated that a ring finger of a hand may be positioned to rest on the button 102 when the hand grips the handle normally. However, in other examples, a button may be positioned for normal contact by the index, middle, ring, and/or small finger.

Because the ratchet button 102 may be located in the normal finger contact area of the lever 82, the ratchet mechanism may be selectively overridden using a normal grip. As a result, no separate ratchet lockout feature is included in this example, although a ratchet lockout feature may be included in other examples.

A ratchet spring 118 may be captured in a slip fit keyway in the ratchet button 102. The ratchet spring 118 may be maintained in its position by the internal walls of the lever 82. The ratchet spring 118 may bias the button 102 to protrude through hole 85 of the lever 82, and at the same time may bias the button friction zone 138 to engage the ratchet link friction zone so that the ratchet mechanism is normally engaged.

The ratchet link 100 can function as an automatic one-way brake or locking device to hold the instrument 70 in a closed or partially closed position without user effort. The ratchet link 100 may resist the action of a biasing member, such as spring 86, which may tend to urge the instrument 70 to an open position. The button 102 can function to temporarily disengage the ratchet link 100 so that the instrument 70 can move toward the open position.

The components disclosed herein may be fabricated from metals, alloys, polymers, plastics, ceramics, glasses, composite materials, or combinations thereof, including but not limited to: PEEK, titanium, titanium alloys, commercially pure titanium grade 2, ASTM F67, Nitinol, cobalt chrome, stainless steel, ultra high molecular weight polyethylene (UHMWPE), biocompatible materials, and biodegradable materials, among others. Different materials may be used for different parts. Different materials may be used within a single part. Any component disclosed herein may be colored, coded or otherwise marked to make it easier for a user to identify the type and size of the component, the setting, the function(s) of the component, and the like.

It should be understood that the present systems, kits, apparatuses, and methods are not intended to be limited to the particular forms disclosed. Rather, they are to cover all combinations, modifications, equivalents, and alternatives falling within the scope of the claims.

The claims are not to be interpreted as including means-plus- or step-plus-function limitations, unless such a limitation is explicitly recited in a given claim using the phrase(s) “means for” or “step for,” respectively.

The term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

In the foregoing Detailed Description, various features are grouped together in several embodiments for the purpose of streamlining the disclosure. This method of disclosure is not to be interpreted as reflecting an intention that the embodiments of the invention require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

1. A handle system comprising:
a handle body comprising an output axis;
a lever comprising a first portion hinged to the handle body at a main pivot joint and a second portion opposite the first portion, wherein the second portion is closer to the output axis than the main pivot joint;
an output point; and
a first link coupling the lever to the output point;
wherein pivoting the lever relative to the handle body moves the output point along the output axis.

2. The handle system of claim 1, wherein the first link is hinged to the lever at a first joint and coupled to the output point.

3. The handle system of claim 2, wherein the first link is hinged to the second portion of the lever.

4. The handle system of claim 2, comprising:
a second link hinged to the first link at a second joint, hinged to the handle body at a third joint, and coupled to the output point, wherein the first and second joints are separated by a first distance, wherein the second and third joints are separated by a second distance.

5. The handle system of claim 4, comprising:
a third link hinged to the second link at a fourth joint and coupled to the output point, wherein the third and fourth joints are separated by a third distance, wherein the second, third, and fourth joints are in a triangular arrangement.

6. The handle system of claim 5, comprising:
a fourth link hinged to the third link at a fifth joint and hinged to the handle body at a sixth joint, wherein the fourth joint comprises the output point, wherein the fourth and fifth joints are separated by a fourth distance, wherein the fifth and sixth joints are separated by a fifth distance.

7. A handle system comprising:
a handle body comprising an output axis;
a lever comprising a first portion hinged to the handle body at a main pivot joint and a second portion opposite the first portion, wherein the second portion is closer to the output axis than the main pivot joint; and
an output point;
wherein the handle system comprises:
a first state, in which the lever is farther from the handle body and the output point is at a first location along the output axis; and
a second state, in which the lever is closer to the handle body and the output point is at a second location along the output axis.

8. The handle system of claim 7, wherein the handle system is biased to remain in the first state until the lever is urged toward the handle body.

9. The handle system of claim 7, comprising:
a one-way brake, wherein the brake prevents the handle system from assuming the first state when the brake is engaged.

10. The handle system of claim 9, wherein the brake is biased to be engaged, wherein the brake is temporarily disengaged by overcoming the bias.

11. The handle system of claim 10, wherein the brake comprises a button, wherein the button protrudes through a hole in the lever, wherein depressing the button temporarily disengages the brake.

12. The handle system of claim 11, wherein, when the handle is grasped by a hand of a user, the output axis is adjacent a thumb of the hand, the main pivot joint is adjacent a small finger of the hand, and a finger of the hand rests on the button.

13. The handle system of claim 12, wherein a ring finger of the hand rests on the button when the handle is grasped by a user.

14. An instrument comprising:
a handle body comprising an output axis;
a lever comprising a first portion hinged to the handle body at a main pivot joint and a second portion opposite the first portion, wherein the second portion is closer to the output axis than the main pivot joint; and
a movable shaft extending from the handle body along the output axis;
wherein the handle system comprises:
a first state, in which the lever is farther from the handle body and the movable shaft is at a first location along the output axis; and
a second state, in which the lever is closer to the handle body and the movable shaft is at a second location along the output axis.
